(19) United States
(12) Patent Application Publication
(10) Pub. No.: US 2022/0175456 A1
(43) Pub. Date: Jun. 9, 2022

(54) SYSTEM AND METHOD FOR ASSISTING ORTHOPEADICS SURGERIES

(71) Applicant: GANYMED ROBOTICS, Paris (FR)

(72) Inventors: Loïc PETER, Zurich (CH); Nicolas LOY RODAS, GENNEVILIERS (FR); Marion DECROUEZ, Sevres (FR)

(73) Assignee: GANYMED ROBOTICS, Paris (FR)

(21) Appl. No.: 17/545,790

(22) Filed: Dec. 8, 2021

(30) Foreign Application Priority Data
Dec. 8, 2020 (EP) 20306516.4

(51) Int. Cl. A61B 34/10

(52) U.S. Cl. CPC A61B 34/10; A61B 2034/104; A61B 2034/2072; A61B 2034/107; A61B 2034/105

(57) ABSTRACT

A system and a computer-implemented method for intra-operatively predicting an outcome of a planar cut performed with a planar surgical tool on a target bone of a subject, wherein the planar surgical tool comprises a planar cutting surface. Also, non-transitory computer readable medium including instructions which, when executed by a computer, cause the computer to carry out the steps of the method.

### FIELD

The present invention pertains to the field of image processing for orthopedic surgery. In particular, the invention relates to a computer-implemented method and system configured to predict an outcome of a planar cut performed with a planar surgical tool on a target bone of a subject during the surgery.

### BACKGROUND

Total knee arthroplasty (TKA) is a surgical procedure to resurface a knee damaged by arthritis. Metal implants are used to cap the ends of the bones that form the knee-joint. During the bone preparation phase, the surgeon uses a surgical cutting tool, usually a sagittal saw to perform 5 planar cuts on the femur and one plane cut on the tibia. In the case of navigated or robotic TKA, a guidance software is used to position the cutting tool within the desired plane with respect to the bone before the cut.

Guidance system are used to align a planar cutting tool relative to a bone being resected according to a target plane. Such systems can guarantee up to a certain precision in the bone cut, however, several factors (patient morphology, a user's familiarity with the system, fiducial registration errors) can still affect their performance and an accurate positioning of the cutting tool may not be fully satisfied. There is no independent way to obtain a qualitative or quantitative information for verifying the accuracy of the bone resection and predicting the anatomical structure's shape after the cut, without actually performing the cut.

The present invention aims to fill this void by providing a method and system for verifying a planar cut and visualizing its future effect before the cut is performed, that can be used in conjunction with robotic and/or navigation systems.

### SUMMARY

The present invention relates to a computer-implemented method for intra-operatively predicting an outcome of a planar cut performed with a planar surgical tool on a target bone of a subject, wherein said planar surgical tool comprises a planar cutting surface, said method comprising:
receiving at least one 3D image previously acquired from at least one 3D imaging sensor; said 3D image being a 3D point cloud comprising at least one portion of the target bone and at least one portion of planar cutting surface of the surgical tool,
segmenting the 3D image obtaining points of the 3D image belonging to the planar cutting surface of the surgical tool and points of the 3D image belonging to the target bone;
obtaining the spatial orientation and position of an osteotomy plane by fitting a plane to the segmented points belonging to the planar cutting surface of the surgical tool;
overlapping the osteotomy plane to the segmented points belonging to the target bone and selecting the points belonging to the portion of target bone intended to be removed with the surgical tool;
outputting the points belonging to the portion of target bone intended to be removed with the surgical tool.

Advantageously, the present method allows to verify the future outcome of a planar cut and visualizing for the medical staff its future effect before the cut is performed, providing therefore a precious information for correcting any alignment error and the like. Furthermore, thanks to an external measurement device (i.e. 3D imaging sensor), this method allows for an independent verification which do not rely on any kinematic or positioning data from the robotic arm or navigation system used during the surgery, thus not affected by their initial positioning errors.

According to one embodiment, the method further comprises generating a simulated 3D model of the removed bone portion using the points belonging to the portion of target bone intended to be removed with the surgical tool.

According to one embodiment, the method further comprises:
receiving a 3D model of the bone and a preoperative planning comprising equations of the planned cutting planes in a reference frame of the 3D model of the bone,
obtaining a planned 3D model of the removed bone portion by applying the equations of the cutting planes to the 3D model of the bone,
comparing the simulated 3D model and said planned 3D model of the removed bone portion using 3D shape analysis and outputting the result of said comparison.

The 3D model of the bone and the simulated 3D model may be 3D digital models as the planned 3D model obtained from the 3D model of the bone.

According to one embodiment, the result of the comparison is a 3D shape similarity measure or a matching error. This output of the method advantageously provides to the medical staff an information on the accuracy of positioning of the surgical tool with respect to the pre-operative planning.

According to one embodiment, the method further comprises calculating principal components of the simulated 3D model, defining from the principal components a bounding box of the removed bone portion and estimating from the bounding box a thickness of the removed bone portion. This embodiment, advantageously allows to calculate the thickness of the bone fragment (i.e., resected bone portion) before actually performing the cut.

According to one embodiment, the method further comprises comparing said estimated thickness of the removed bone portion to a planned thickness of the removed bone portion and outputting an alert whenever a deviation from the planned thickness is detected. This advantageously alert the medical staff that a correction on the positioning of the surgical tool may be performed.

According to one embodiment, the surgical tool comprises a fiducial marker to help obtaining the spatial orientation and position of the osteotomy plane.

According to one embodiment, the target bone is a femur or a tibia.

The present invention also relates to a computer program comprising instructions which, when executed by a computer, cause the computer to carry out the steps of the method according to any one of the embodiments described above.

The present invention also relates to a computer readable medium comprising instructions which, when executed by a computer, cause the computer to carry out the steps of the method according to any one of the embodiments described above.

The present invention also relates to a system for intra-operatively predicting an outcome of a planar cut performed with a planar surgical tool on a target bone of a subject, wherein the planar surgical tool comprises a planar cutting surface, said system comprising:
at least one input adapted to receive at least one 3D image previously acquired from at least one 3D imaging sensor, said 3D image being a 3D point cloud comprising at least one portion of the target bone and at least one portion of the planar cutting surface of the surgical tool,
at least one processor configured to:
   segment the 3D image obtaining points of the 3D image belonging to the planar cutting surface of the surgical tool and points of the 3D image belonging to the target bone;
   obtain the spatial orientation and position of an osteotomy plane by 3D fitting the segmented points belonging to the planar cutting surface of the surgical tool;
   overlap the osteotomy plane to the segmented points belonging to the target bone and selecting the points belonging to the portion of target bone intended to be removed with the surgical tool;
at least one output adapted to provide the points belonging to the portion of target bone intended to be removed with the surgical tool.

According to one embodiment, the processor is further configured to generate a simulated 3D model of the removed bone portion using the points belonging to the portion of target bone intended to be removed with the surgical tool.

According to one embodiment, the processor is further configured to:
receive a 3D model of the bone and a preoperative planning comprising equations of the cutting planes in a reference frame of the 3D model of the bone,
obtain a planned 3D model of the removed bone portion by applying the equations of the cutting planes to the 3D model of the bone,
compare the simulated 3D model and said planned 3D model of the removed bone portion using 3D shape analysis and outputting the result of said comparison.

According to one embodiment, the result of the comparison is a 3D shape similarity measure or a matching error.

According to one embodiment, the processor is further configured to calculate principal components of the simulated 3D model, defining from the principal components a bounding box of the removed bone portion and estimating from the bounding box a thickness of the removed bone portion.

According to one embodiment, the processor is further configured to compare said estimated thickness of the removed bone portion to a planned thickness of the removed bone portion and outputting an alert whenever a deviation from the planned thickness is detected.

In the present invention, the following terms have the following meanings:
“bone resection thickness” refers to the smaller dimension of the resected portion of the bone.
“adapted” and “configured” are used in the present disclosure as broadly encompassing initial configuration, later adaptation or complementation of the present device, or any combination thereof alike, whether effected through material or software means (including firmware).
“processor” should not be construed to be restricted to hardware capable of executing software, and refers in a general way to a processing device, which can for example include a computer, a microprocessor, an integrated circuit, or a programmable logic device (PLD). The processor may also encompass one or more Graphics Processing Units (GPU), whether exploited for computer graphics and image processing or other functions. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.
“reference frame” refers to a coordinate system that uses one or more numbers, or coordinates, to uniquely determine the position of the points or other geometric elements on a manifold such as Euclidean space.
“tridimensional digital model” refers to a three-dimensional digital (or virtual) model being a virtual object in 3 dimensions. The position and orientation of the model is known in the associated digital referential.
“preoperative planning” in the context of surgery, refers to a list of actions to be performed during the different surgical phases. This surgical planning may be obtained by means of a simulation program carried out before the operation which uses a 3-dimensional digital model of the bone(s) of the patient that are the target of the surgery. In the case of a knee arthroplasty operation, for example, pre-operative planning will consist of defining each of the cutting planes and drilling axes in relation to a three-dimensional model of the femur and tibia.

The following detailed description will be better understood when read in conjunction with the drawings. For the purpose of illustrating, the steps of the method are shown in the preferred embodiments. It should be understood, however that the present invention is not limited to the precise arrangements, structures, features, embodiments, and aspect shown. The drawings are not drawn to scale and are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

The functions of the various elements shown in the figures may be provided through the use of dedicated hardware as well as hardware capable of executing software in association with appropriate software. When provided by a processor, the functions may be provided by a single dedicated processor, a single shared processor, or a plurality of individual processors, some of which may be shared.

It should be understood that the elements shown in the figures may be implemented in various forms of hardware, software or combinations thereof. Preferably, these elements are implemented in a combination of hardware and software on one or more appropriately programmed general-purpose devices, which may include a processor, memory and input/output interfaces.

### BRIEF DESCRIPTION OF THE DRAWINGS

Features and advantages of the invention will become apparent from the following description of embodiments of a system, this description being given merely by way of example and with reference to the appended drawings in which:

FIG. 1 is a block diagram representing the main steps of the method according to one embodiment of the present invention.

FIG. 2 is a schematic representation of a color image of the target bone and the planar surgical tool positioned as before a planar cut is performed.

FIG. 3 is a depth image corresponding to the color image of FIG. 2.

FIG. 4 shows (a) the point clouds belonging to both the planar cutting surface of the surgical tool and the target bone after segmentation, (b) the osteotomy plane overlapping the point clouds belonging to the planar cutting surface of the surgical tool and the target bone after segmentation and (c) the points belonging to the portion of target bone intended to be removed with the surgical tool.

FIG. 5 shows the planned 3D model of the removed bone portion and of the resected bone.

FIG. 6 shows the tibia after the performance of the planar cuts on the right and with the adequate prosthesis on the left.

FIG. 7 shows the femur after the performance of the planar cuts on the right and with the adequate prosthesis on the left.

FIG. 8 shows the bone part remove after an antero femoral cut and the thickness of the removed part measured with a sliding caliper.

### DETAILED DESCRIPTION

While various embodiments have been described and illustrated, the detailed description is not to be construed as being limited hereto. Various modifications can be made to the embodiments by those skilled in the art without departing from the true spirit and scope of the disclosure as defined by the claims.

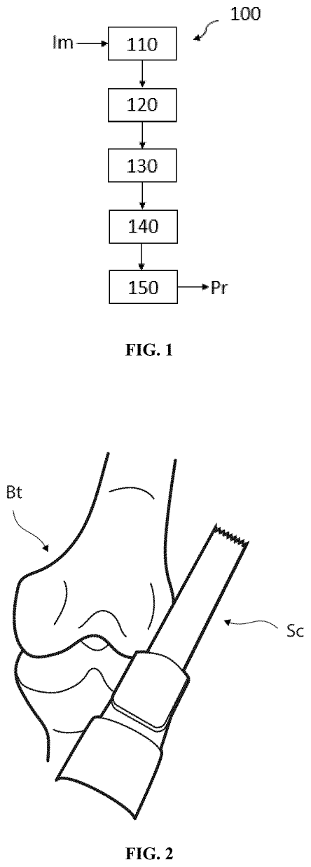

As shown in FIG. 1, a first aspect of the present invention concerns a computer-implemented method 100 comprising multiple steps aiming to predict intra-operatively an outcome of at least one planar cut performed with a planar surgical tool on a target bone Bt of a subject.

FIG. 2 shows a schematic representation of the target bone Bt with the planar surgical tool positioned so that the planar cutting surface Sc has a spatial orientation and position adapted to perform the planar cut according to a preoperative planning. The surgical tool may be mounted on a kinematic chain that is configured to position the surgical tool according to the preoperative planning.

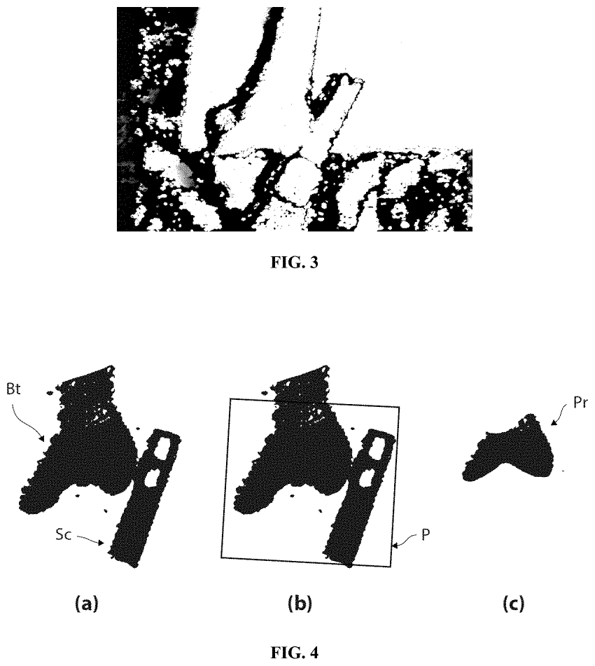

According to the embodiment in FIG. 1, the first step 110 of the method consists in receiving at least one 3D image comprising at least one portion of the target bone Bt and at least one portion of planar cutting surface Sc of the surgical tool which has been acquired from at least one 3D imaging sensor before performing the cut on the target bone Bt. FIG. 3 shows the depth image of the scene represented in FIG. 2.

The 3D image obtained from the 3D imaging sensor comprises information of the distance between each point of the scene acquired in the 3D image and the 3D imaging sensor.

Therefore, the raw 3D image obtained by the 3D imaging sensor is also called a depth map, or depth image that may be presented under the form of a bidimensional array representing a grey level image or a RGB image, wherein the size of the array depends on the camera type and sensor dimensions.

According to one embodiment, the acquisition of the 3D image is carried out using at least two cameras to carry out an acquisition by stereovision. According to another embodiment, a projector is used to project a pattern (textured light) on the scene that will help the matching of the data streamed from the two cameras.

The use of a 3D imaging sensor advantageously allows to obtain information of the bone surface in an easy, non-invasive and fast way since one image captures all the surgical field, without coming into contact with the patient (as for palpation techniques).

The at least one 3D imaging sensor 30 may have a fixed position with respect to the target 10 in the surgical theatre or alternatively it may be kinematically linked to the surgical navigation or robotic system.

The method may further comprise a pre-processing step implementing noise reduction algorithms.

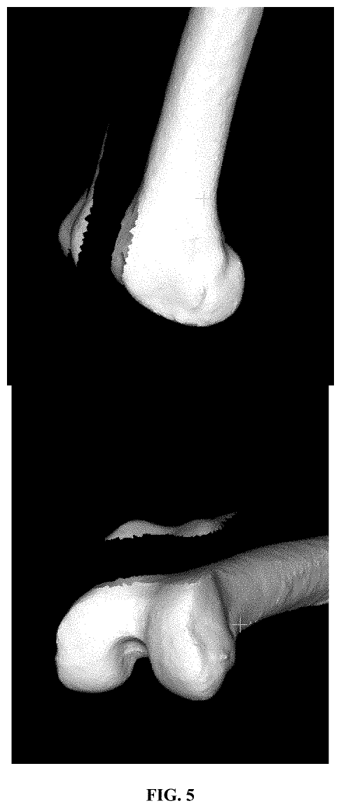

According to one embodiment, the second step 120 comprises segmenting the point cloud of the 3D image so as to obtain one point cloud comprising the points belonging to the planar cutting surface of the surgical tool Sc and one point cloud comprising the point of the target bone Bt. FIG. 4a shows the point clouds belonging to the planar cutting surface of the surgical tool Sc and the target bone Bt.

As shown in FIG. 4b, in one embodiment, the following step 130 comprises obtaining the spatial orientation and position of an osteotomy plane P by fitting a plane to the segmented points belonging to the planar cutting surface of the surgical tool Sc.

In one embodiment, the step 140 of the method comprises selecting the points belonging to the portion of target bone intended to be removed with the surgical tool Pr by overlapping 140 the osteotomy plane P to the segmented points belonging to the target bone Bt. FIG. 4c shows the point cloud points belonging to the portion of target bone intended to be removed Pr that is obtained at step 140. This advantageously allows the segmentation of the portion of target bone intended to be removed as it would be if the planar cut was performed along the suggested plane P.

In one embodiment, the method comprises the step 150 of outputting the points belonging to the portion of target bone intended to be removed with the surgical tool Pr. The output may simply be an information transferred to at least one data storage medium or database or to a digital to analogue module. Said digital to analogue module may be a display to visualize the portion of target bone intended to be removed Pr.

According to one embodiment, the method further comprises generating a simulated 3D model of the removed bone portion by rendering the points belonging to the portion of target bone intended to be removed with the surgical tool.

According to one embodiment, the method comprises retrieving from a computer readable storage medium, a server or the like a 3D model of the target bone Bt to be treated during the surgery using the surgical tool. Said 3D model is a three-dimensional virtual representation of the target bone Bt.

In one embodiment, the 3D model of the target bone Bt is generated using imaging data acquired using computed tomography or MRI systems. Other imaging techniques may be as well used such as X-rays, fluoroscopy, ultrasound or other imaging means. In this case, the three-dimensional digital model is obtained previous to the surgery.

In one embodiment, the 3D model of the target bone Bt is generated using 2D X-ray radiographies comprising the target, a statistical shape model of the target and/or the 3D image acquired intraoperatively by the 3D imaging sensor. This embodiment advantageously allows to generate a three-dimensional model even when the 3D imaging data (i.e. computed tomography or MRI) are not available.

In one embodiment, the 3D model of the target bone Bt is modified to simulate measurement noise or the presence of cartilage. Said modifications may be calculated from training data or biomechanical simulation data.

Together with the 3D model the method is configured to receive a preoperative planning which may be as well retrieved from a computer readable storage medium or a medical server. Said preoperative planning comprises the equations of the cutting planes in the same reference frame of the bone model. The equations of the cutting planes may then be used to obtain a planned 3D model of the removed bone portion by applying the cutting planes to the 3D model of the bone. Afterwards, the simulated 3D model and said planned 3D model of the removed bone portion are compared using 3D shape analysis. The result of this comparison may be a matching error or a 3D shape similarity measure. The matching error may be implemented by matching of the 3D models using ICP based methods (Iterative Closest Point) and inferring the angular deviation between the simulated cutting plane and the planned cutting plane. For the shape similarity measure shape metrics like the Hausdorff distance or the Jaccard distance may be used after the matching. Finally, the result of said comparison is provided as output. FIG. 5 shows an example of visualization of the planned 3D model of the removed bone portion.

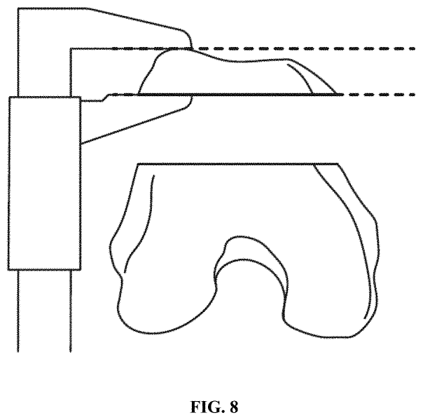

According to one embodiment, the method further comprises calculating principal components of the simulated 3D model and defining from the principal components a bounding box of the removed bone. One side of the bounding box coincides with the simulated osteotomy plane. The bounding box is the used to estimate the thickness of the removed bone portion. This step advantageously allows to calculate the thickness of the bone fragment (i.e. resected bone portion) before actually performing the cut. FIG. 8 provides an illustrative representation of the thickness of the bone fragment that this method allows to calculate before the intervention and which may be measured with a sliding caliper after the surgical resection.

According to one advantageously embodiment, the method comprises comparing the estimated thickness of the removed bone portion to a planned thickness of the removed bone portion and outputting an alert whenever a deviation is detected so that the medical staff is informed that there is a discrepancy between the preoperative planning cutting planes and the actual position of the surgical tool.

In one embodiment, the surgical tool comprises a fiducial marker visible with the imaging sensor to help the estimation of the osteotomy plane. Depending on the imaging sensor, those markers can be very easy to detect in the streamed data. In the present application, infrared reflective marker may be used with a stereo vision sensor with infrared sensors or contrasted black and white marker, like Aruco markers, may be used with RGB-D cameras. Advantageously, the fiducial markers are easy to detect in the images so that it is easier to segment the points belonging to the planar surgical tool. Therefore, using the fiducial markers, the segmented points belonging to the planar cutting surface of the surgical tool contains less or no outliers and as consequence the equation of the osteotomy plane is more accurate.

Advantageously, the information obtained with the method of the present invention allows for independent verification of the preoperative planning realization.

According to one embodiment, the method comprises the visualization of the outputs of the different steps in real time. Notably, the visualization is configured to show in real time the surface of the simulated 3D model of the removed bone portion at the same time of the surface of the planned 3D model of the removed bone portion. These two models may be overlapped in a consistent manner (principal component alignment and/or registration of the 3D models) and the visualization may be configured to highlight the differences between them. These overlapped models may be displayed simultaneously to the values obtained from their comparison using 3D shape analysis so as to provide both qualitative and quantitative feedback to the user. Using such a visual feedback, the surgeon can advantageously adapt the surgical tool position to minimize the differences so that the surface of the simulated 3D model of the removed bone portion matches the surface of the planned 3D model of the removed bone portion.

The method may be used in conjunction with a navigation system. In this case, the planar surgical tool and the portion of target bone intended to be removed with the surgical tool are computed independently from the navigation system and therefore do not rely on it. Advantageously, the method can provide an independent verification and assessment of the projected bone resection accuracy.

The method may as well be used in conjunction with a robotic system. In that case, the method does not use the knowledge of the surgical tool's position computed from the robot's kinematic and sensors. This way, the method can advantageously provide an independent verification and assessment of the projected bone resection accuracy.

The method of the present invention may be used for the target bone being the tibia or the femur.

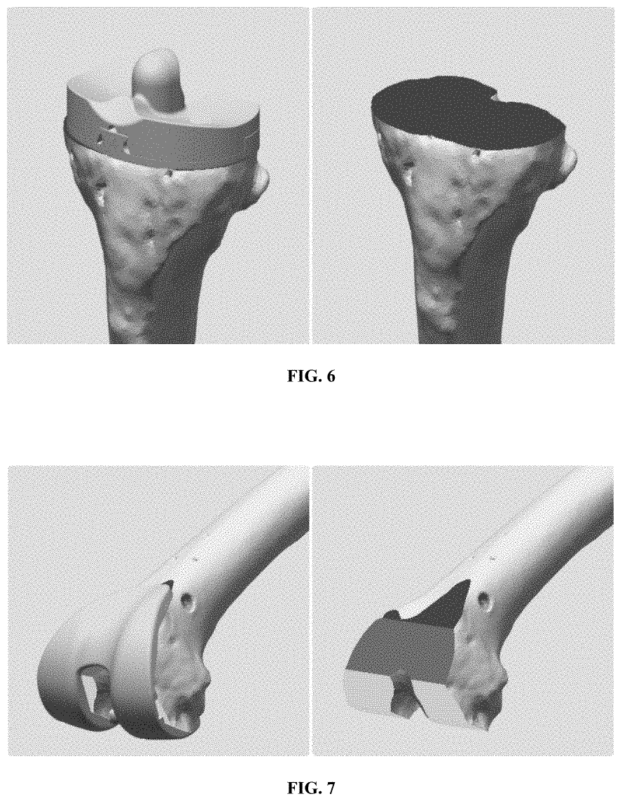

An example of the tibia after performing one cut with and without the implant is shown in FIG. 6.

According to one embodiment, the steps 110 to 150 are repeated each time that the surgical tool is repositioned with respect to the target bones according to a new cutting plane comprised in the preoperative planning. Indeed, as in the example of the femur shown in FIG. 7, the femur is cut according to 5 planes in order to correctly fit the implant.

The present invention also relates to the system comprising means to carry out the steps of the method described above. Notably, the system of the present invention comprises
at least one input adapted to receive at least one 3D image previously acquired from at least one 3D imaging sensor, said 3D image being a 3D point cloud comprising at least one portion of the target bone and at least one portion of the planar cutting surface of the surgical tool,
at least one processor configured to:
   segmenting the points of the 3D image belonging to the planar cutting surface of surgical tool and the target bone;
   obtaining the spatial orientation and position of an osteotomy plane by 3D fitting the segmented points belonging to the planar cutting surface of the surgical tool;
   overlapping the osteotomy plane to the segmented points belonging to the target bone and selecting the points belonging to the portion of target bone intended to be removed with the surgical tool;
at least one output adapted to provide the points belonging to the portion of target bone intended to be removed with the surgical tool.

In another embodiment, the system is integrated in the robot guidance software which automatically corrects the saw trajectory in order to guarantee that the cutting plane matches the surgical planning.

Embodiments disclosed herein include various operations implemented in the different steps of the method as described in this specification. As discussed above, the operations may be performed by hardware components and/or may be embodied in machine-executable instructions, which may be used to cause a general-purpose or special-purpose processor programmed with the instructions to perform the operations. Alternatively, the operations may be performed by a combination of hardware, software, and/or firmware.

The performance of one or more operations described herein may be distributed among one or more processors, not only residing within a single machine, but deployed across a number of machines. In some examples, the one or more processors or processor-implemented modules may be located in a single geographic location (e.g., within a home environment, an office environment, or a server farm). In other embodiments, the one or more processors or processor-implemented modules may be distributed across a number of geographic locations.

The present invention further comprises a computer program product for intra-operatively predicting an outcome of a planar cut, the computer program product comprising instructions which, when the program is executed by a computer, cause the computer to carry out the steps of the method according to any one of the embodiments described hereabove.

The computer program product to perform the method as described above may be written as computer programs, code segments, instructions or any combination thereof, for individually or collectively instructing or configuring the processor or computer to operate as a machine or special-purpose computer to perform the operations performed by hardware components. In one example, the computer program product includes machine code that is directly executed by a processor or a computer, such as machine code produced by a compiler. In another example, the computer program product includes higher-level code that is executed by a processor or a computer using an interpreter. Programmers of ordinary skill in the art can readily write the instructions or software based on the block diagrams and the flow charts illustrated in the drawings and the corresponding descriptions in the specification, which disclose algorithms for performing the operations of the method as described above.

The present invention further comprises a computer-readable storage medium comprising instructions which, when the program is executed by a computer, cause the computer to carry out the steps of the method according to any one of the embodiments described hereabove.

According to one embodiment, the computer-readable storage medium is a non-transitory computer-readable storage medium.

Computer programs implementing the method of the present embodiments can commonly be distributed to users on a distribution computer-readable storage medium such as, but not limited to, an SD card, an external storage device, a microchip, a flash memory device, a portable hard drive and software websites. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

### EXAMPLES

The present invention is further illustrated by the following practical example wherein the method and system of the present invention are applied to robotic total knee arthroplasty (TKA) procedures and especially to the preparation of the femoral surface.

At the pre-operative stage, a CT-scan of the lower limb of the patient is performed. The data is segmented to obtain a 3D model of the femur (i.e. target bone). The implant 3D model is placed on the femur 3D model using a dedicated planning software. Thus, the equations of the planar cuts are defined in the coordinate system of the femur 3D model.

During the bone preparation phase and after the femur exposition, the planar cutting tool is positioned within the planned osteotomy plane as comprised in the preoperative planning estimated by the guidance software with uncertainty in the accuracy. A depth camera images the exposed femur and the saw. The step 120 of the method is used to detect and segment the depth data belonging to the femur and the saw. Thanks to the planarity of the planar cutting surface of the saw, the osteotomy plane is estimated by fitting a 3D plane to the segmented point cloud belonging to the saw. Given the equation of the osteotomy plane, the depth data belonging to the femur is segmented by selecting the points belonging to the portion of target bone intended to be removed with the surgical tool.

Thus, the method allows to obtain a 3D representation of a simulated removed part that would be obtained if the cut were performed following the preoperative planning. The simulated bone fragment is then compared to the planned bone fragment from the preoperative stage through 3D shape analysis. The surgeon can visualize the simulated bone fragment, the simulated resected bone and the comparison with the preoperative planning on a dedicated graphical user interface displayed on a screen.

This system and method allow to compare, right before actually performing the cut (which is irreversible), whether the simulated 3D model of the removed bone portion matches the preoperative planning. The system also provides metrics to quantitively assess the similarity between the simulated and planned 3D model of the removed bone portion. This comparison does not depend on the guidance system itself (robotic arm for instance) and therefore is a separate way of checking that the surgical execution is accurate.

Surgical guidance systems also often allow to visualize the osteotomy plane before cutting (displayed on the screen or using augmented reality). However, this feature uses the exact same information that is used to position the saw in the first place, and not more. Therefore, if an error was already present in the surgical tool positioning (namely if the robot has registered that it is positioned in point A when it is, in fact, it is in point B), then the same error will be reproduced in the pre-cut verification feature.

A typical robotic system for orthopedic surgery, and TKA in particular, uses planning, initial registration and embedded sensors to direct the physical positioning of the surgical tool in space relative to the bone to be resected. Using the same information to check whether the surgical tool is properly positioned therefore does not prevent the error either.

In contrast, the present system and method advantageously allow for an independent verification relying on an external measurement device (depth camera), which do not rely on any kinematic or positioning data from the robotic or navigation system, thus not affected by their initial positioning errors.

1. A computer-implemented method for intra-operatively predicting an outcome of a planar cut performed with a planar surgical tool on a target bone of a subject, wherein said planar surgical tool comprises a planar cutting surface, said method comprising:
receiving at least one 3D image previously acquired from at least one 3D imaging sensor; said 3D image being a 3D point cloud comprising at least one portion of the target bone and at least one portion of planar cutting surface of the surgical tool,
segmenting the 3D image obtaining points of the 3D image belonging to the planar cutting surface of the surgical tool and points of the 3D image belonging to the target bone;
obtaining the spatial orientation and position of an osteotomy plane by fitting a plane to the segmented points belonging to the planar cutting surface of the surgical tool;
overlapping the osteotomy plane to the segmented points belonging to the target bone and selecting the points belonging to the portion of target bone intended to be removed with the surgical tool; and
outputting the points belonging to the portion of target bone intended to be removed with the surgical tool (Pr).

2. The method according to claim 1, further comprising generating a simulated 3D model of the removed bone portion using the points belonging to the portion of target bone intended to be removed with the surgical tool.

3. The method according to claim 2, further comprising:
receiving a 3D model of the bone and a preoperative planning comprising equations of the planned cutting planes in a reference frame of the 3D model of the bone,
obtaining a planned 3D model of the removed bone portion by applying the equations of the cutting planes to the 3D model of the bone,
comparing the simulated 3D model and said planned 3D model of the removed bone portion using 3D shape analysis and outputting the result of said comparison.

4. The method according to claim 3, wherein the result of the comparison is a 3D shape similarity measure or a matching error.

5. The method according to claim 2, further comprising calculating principal components of the simulated 3D model, defining from the principal components a bounding box of the removed bone portion and estimating from the bounding box a thickness of the removed bone portion.

6. The method according to claim 5, further comprising comparing said estimated thickness of the removed bone portion to a planned thickness of the removed bone portion and outputting an alert whenever a deviation from the planned thickness is detected.

7. The method according to claim 1, wherein the surgical tool comprises a fiducial marker to help obtaining the spatial orientation and position of the osteotomy plane.

8. The method according to claim 1, wherein the target bone is a femur or a tibia.

9. A non-transitory computer readable medium comprising instructions which, when executed by a computer, cause the computer to carry out the steps of the method according to claim 1.

10. A system for intra-operatively predicting an outcome of a planar cut performed with a planar surgical tool on a target bone of a subject, wherein the planar surgical tool comprises a planar cutting surface, said system comprising:
at least one input adapted to receive at least one 3D image previously acquired from at least one 3D imaging sensor, said 3D image being a 3D point cloud comprising at least one portion of the target bone and at least one portion of the planar cutting surface of the surgical tool,
at least one processor configured to:
segment the 3D image obtaining points of the 3D image belonging to the planar cutting surface of the surgical tool and points of the 3D image belonging to the target bone;
obtain the spatial orientation and position of an osteotomy plane by 3D fitting the segmented points belonging to the planar cutting surface of the surgical tool; and
overlap the osteotomy plane to the segmented points belonging to the target bone and selecting the points belonging to the portion of target bone intended to be removed with the surgical tool; and
at least one output adapted to provide the points belonging to the portion of target bone intended to be removed with the surgical tool.

11. The system according to claim 10, wherein the at least one processor is further configured to generate a simulated 3D model of the removed bone portion using the points belonging to the portion of target bone intended to be removed with the surgical tool.

12. The system according to claim 10, wherein the at least one processor is further configured to:
receiving a 3D model of the bone and a preoperative planning comprising equations of the planned cutting planes in a reference frame of the 3D model of the bone,
obtaining a planned 3D model of the removed bone portion by applying the equations of the cutting planes to the 3D model of the bone,
comparing the simulated 3D model and said planned 3D model of the removed bone portion using 3D shape analysis and outputting the result of said comparison.

13. The system according to claim 11, wherein the at least one processor is further configured to calculating principal components of the simulated 3D model, defining from the principal components a bounding box of the removed bone portion and estimating from the bounding box a thickness of the removed bone portion.

14. The system according to claim 13, wherein the at least one processor is further configured to comparing said estimated thickness of the removed bone portion to a planned thickness of the removed bone portion and outputting an alert whenever a deviation from the planned thickness is detected.

15. The system according to claim 12, wherein the at least one processor is further configured to generate as result the result of the comparison between the simulated 3D model and said planned 3D model a 3D shape similarity measure or a matching error.
